(19) United States
(12) Patent Application Publication
(10) Pub. No.: US 2009/0105484 A1
(43) Pub. Date: Apr. 23, 2009

(54) PROCESS FOR THE PREPARATION OF ARYL SUBSTITUTED OXAZOLIDINONES AS INTERMEDIATES FOR ANTIBACTERIAL AGENTS

(75) Inventors: Janette Cherryman, Cheshire (GB); Murray Watson Cuthbert, Cheshire (GB); Jerome Dubiez, Cheshire (GB); Garnet Edward Howells, Cheshire (GB); Keith Raymond Mulholland, Cheshire (GB)

Correspondence Address: MORGAN LEWIS & BOCKIUS LLP, 1111 PENNSYLVANIA AVENUE NW, WASHINGTON, DC, 20004, US

(73) Assignee: AstraZeneca, Sodertajle (SE)

(21) Appl. No.: 11/569,399

(22) PCT Filed: May 24, 2005
(86) PCT No.: PCT/GB05/02042

§ 371 Date: Nov. 20, 2006

(30) Foreign Application Priority Data
May 25, 2004 (GB) 0411596.0

(51) Int. Cl. C07D 263/04

(52) U.S. Cl. 548/229

(57) ABSTRACT

A compound of the formula (VIII) wherein each X is independently H or F; and R is selected from hydrogen, halogen, cyano, methyl, cyanomethyl, fluoromethyl, difluoromethyl, trifluoromethyl and —Si[(1-4C)alkyl]₃; and processes for preparing a compound of formula (VIII) by bromination of a compound of the formula (VII),

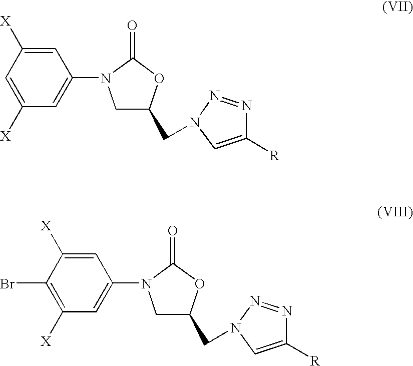

Oxazolidinone containing antibacterial agents have been known for over twenty years. A core feature of the majority of these agents comprises a (substituted) phenyl group attached to the nitrogen of the oxazolidinone ring. Many of these phenyl groups have one or two halo substituents, particularly fluoro, in the meta position(s) relative to the point of attachment to the oxazolidinone ring.

An example of such a structure is a compound of formula (I), where each X is H or halo, for example fluoro; R¹ is often a group such as acetamide, or a C- or N-linked heterocycle (such as triazolyl) and R² may be a wide range of substituents, as described for example in WO 01/81350.

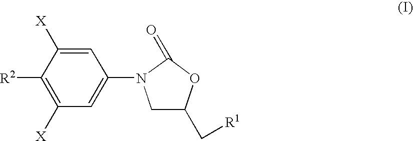

Subsequently, our patent application WO 03/022824 disclosed compounds containing a bi-aryl group (where each aryl group could be phenyl or one of a selection of heteroaryl rings) and two oxazolidinone and/or isoxazoline rings, such as (II) below:

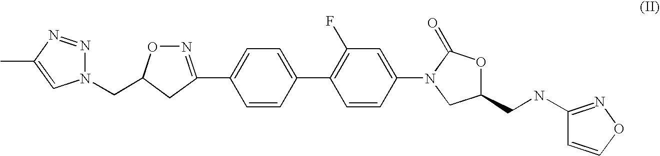

An efficient, convergent approach to the synthesis of such bi-aryl compounds utilises formation of the aryl-aryl bond as a key synthetic step. For this approach, an intermediate of formula (III) (wherein Y is a group suitable to allow further reaction to form the rest of the target compound) is required.

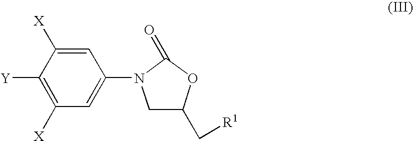

In our patent application, WO 03/022824, halo derivatives of formula (III) above, where R¹ is for example a triazole ring, were converted into corresponding tin derivatives and then coupled as shown in Scheme 1 below.

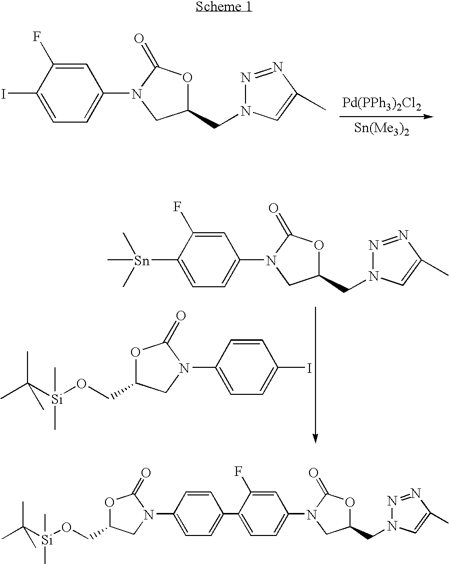

Other suitable derivatives for such coupling reactions are compounds of the formula (III) wherein Y is a boron derivative such as a boronate ester, which are themselves obtainable from halo compounds.

Our co-pending applications PCT/GB2003/005087 and PCT/GB2004/000730 describe further examples of bi-aryl antibacterial agents, the majority of which have a triazole or substituted triazole ring attached to the oxazolidinone ring. The most common coupling reaction in these applications uses a boronate ester such as (IV), which is made from an iodo derivative such as (V):

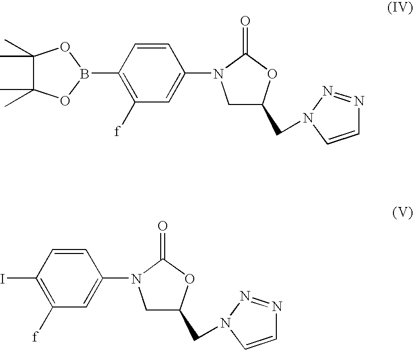

However, the use of iodine-containing compounds on a manufacturing scale is generally considered undesirable, for example for environmental reasons, and therefore it would be preferable to use an alternative halo substituent, such as a bromo substituent.

Therefore an efficient approach to intermediates such as (VI) (and the difluoro, des-fluoro and substituted triazole analogues thereof) is required.

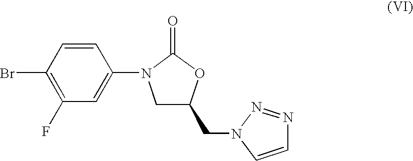

Surprisingly we have found that it is possible to introduce a bromine substituent, required for making the tin or boron reagents, after formation of the triazole ring, without causing side reactions such as bromination of the triazole ring.

According to a first aspect of the invention there is provided a process for forming a compound of the formula (VII) from a compound of the formula (VII), wherein each X is independently H or F and R is selected from hydrogen, halogen, cyano, methyl, cyanomethyl, fluoromethyl, difluoromethyl, trifluoromethyl and —Si[(1-4C)alkyl]₃;

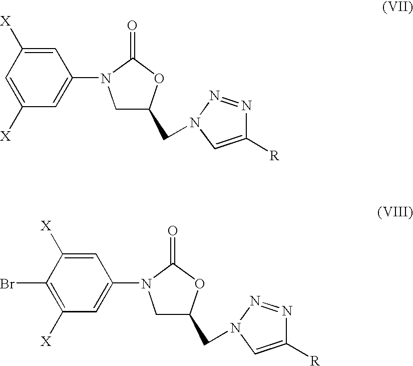

said process comprising treatment of a solution of the compound of formula (VU) with bromine.

It will be appreciated that solutions of bromine convenient for use in such reactions will tend to degrade with time, such that the concentration of bromine present will decrease and render the reagent unsuitable for use. It will be appreciated that for large scale manufacturing, for example in the manufacture of pharmaceutical agents, quality control procedures demand that the concentration of regents is known and controlled within a specified range. Therefore, a more convenient way to provide bromine for such a reaction is to produce bromine in the reaction medium, for example by the reaction between a bromate, a bromide and acid, according to the reaction:

BrO₃-+6H⁺+5Br-→3Br₂+3H₂O

According to a further aspect of the invention, there is provided a process for forming a compound of the formula (VIII) from a compound of the formula (VII) as hereinbefore defined, wherein the bromine is generated in situ from a bromate, a bromide and acid.

According to a further aspect of the invention, there is provided a process for forming a compound of the formula (VIII) from a compound of the formula (VII) as hereinbefore defined, said process comprising treatment of a solution of the compound of formula (VII) with a bromate, a bromide and acid.

Conveniently, the acid and bromide may be provided together by use of hydrobromic acid. Suitably the bromide is added as a solution in water, for example an aqueous solution of hydrobromic acid, such as a 48% w/w aqueous hydrobromic acid solution. Any convenient concentration of such a solution may be used.

Conveniently the bromate is an alkali metal bromate, such as potassium bromate or sodium bromate. Suitably the bromate is added as a solution in water.

The compound of formula (VII) may be dissolved in any suitable organic solvent. In this context, suitable means that the organic solvent must be miscible with water and must not react with the other reagents.

A suitable solvent is acetic acid. The compound of formula (VIII) may be dissolved in a mixture of said suitable organic solvent, such as acetic acid, and water.

Conveniently, the aqueous solution of bromide is added to the solution of the compound of formula (VII), then the solution of bromate is added.

The reaction between bromate and bromide in the presence of acid is exothermic. Conveniently, a vessel containing the reaction mixture may be cooled, for instance in an ice-bath, but maintenance at a particular temperature is not essential for the yield or quality of the product produced. Conveniently a vessel containing the reaction mixture is cooled in an ice-bath such that the temperature of the reaction ranges between 10 and 30° C. during the addition of bromate.

Suitably slight molar excesses of bromate and bromide are used in comparison to the quantity of the compound of formula (VII) used. Suitable amounts of bromate and bromide are those used in the accompanying Examples.

The rate of addition of the bromate solution is not critical. Conveniently, it is added at a rate such that the temperature of the reaction is maintained between 10 and 30° C. during the addition of bromate.

The reaction mixture may be stirred, for example at about ambient temperature, until the reaction is complete. Typically, the reaction may take 3-4 hours to complete, including the time required for addition of bromate.

After the reaction is complete, it is desirable to remove any excess bromine generated before isolation of the product. Conveniently this may be achieved by addition of a solution of metabisulfite, for example a solution of sodium metabisulfite in water. Sufficient metabisulfite is added to react with any residual bromine.

The product may be isolated by any convenient means, for example by filtration from the reaction mixture, or by dissolution into another organic solvent and appropriate washing and evaporation. If the product solidifies from the reaction mixture, it may be convenient to re-dissolve it (for example by heating the solution, for example to about 80-85° C.) and allow crystallisation in a controlled manner.

According to a further aspect of the invention, there is provided a process for forming a compound of the formula (VIII) from a compound of the formula (VII) as hereinbefore defined, said process comprising treatment of a solution of the compound of formula (VII) with an alkali metal bromate, and hydrobromic acid.

According to a further aspect of the invention, there is provided a process for forming a compound of the formula (VIII) from a compound of the formula (VII) as hereinbefore defined, said process comprising:

a) treatment of a solution of the compound of formula (VII) in a mixture of water and a suitable organic solvent with aqueous hydrobromic acid; and
b) addition of an aqueous solution of an alkali metal bromate.

According to a further aspect of the invention, there is provided a process for forming a compound of the formula (VIII) from a compound of the formula (VII) as hereinbefore defined, said process comprising:

a) treatment of a solution of the compound of formula (VII) in a mixture of water and a suitable organic solvent with aqueous hydrobromic acid;
b) addition of an aqueous solution of an alkali metal bromate; and
c) addition of a solution of sodium metabisulfite to react with any excess bromine.

According to a further aspect of the invention, there is provided a process for forming a compound of the formula (VIII) from a compound of the formula (VII) as hereinbefore defined, said process comprising:

a) treatment of a solution of the compound of formula (VII) in a mixture of water and a suitable organic solvent with aqueous hydrobromic acid;
b) addition of an aqueous solution of an alkali metal bromate;
c) addition of a solution of sodium metabisulfite to react with any excess bromine;
d) isolation of the product compound of the formula (VIII).

According to a further aspect of the invention, there is provided a process for forming a compound of the formula (VIII) from a compound of the formula (VII) as hereinbefore defined, said process comprising:

a) treatment of a solution of the compound of formula (VII) in a mixture of water and a suitable organic solvent with aqueous hydrobromic acid;
b) addition of an aqueous solution of an alkali metal bromate;
c) addition of a solution of sodium metabisulfite to react with any excess bromine;
d) isolation of the product compound of the formula (VIII) by heating the mixture resulting from step c) until any solid has dissolved and then cooling the solution until the compound of the formula (VIII) crystallises.

Compounds of the formula (VIII), wherein X and R are as hereinbefore defined are novel, and comprise an independent aspect of the invention. Particular such compounds are those wherein R is hydrogen, halogen or methyl; further particular compounds are those wherein R is hydrogen or methyl, most particularly hydrogen. A preferred compound of the invention is the compound of formula (VI).

In one aspect of the invention, in the compound of formula (VII) at least one X is F. In another aspect, both X are F.

In one aspect, of the invention, in the compound of formula (VII), R is selected from hydrogen, halogen, cyano, methyl, cyanomethyl, fluoromethyl, difluoromethyl, trifluoromethyl and —Si](1-4C)alkyl]₃. Suitable values for R as —Si[(1-4C)alkyl]₃ are trimethylsilyl, triethylsilyl, tert-butyldimethylsilyl and triisopropylsilyl. Further suitable values for R as —Si[(1-4C)alkyl]₃ are trimethylsilyl, triethylsilyl and triisopropylsilyl. In a further aspect, R is hydrogen or methyl. In a still further aspect, R is hydrogen.

The process of the invention is useful in the preparation of compounds of the formula (A),

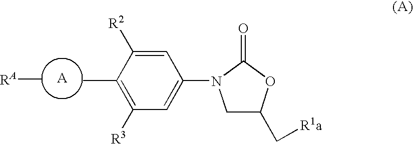

wherein

### R¹ a is

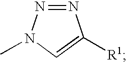

R² and R³ are independently selected from hydrogen and fluorine;
R¹ is selected from hydrogen, halogen, cyano, methyl, cyanomethyl, fluoromethyl, difluoromethyl, trifluoromethyl and —Si[(1-4C)alkyl]₃;
A is an aryl or heteroaryl ring; and
R^{A} is any group commonplace in the antibacterial oxazolidinone art.

Preferably, A is phenyl, pyridyl, pyrimidinyl or thienyl. More preferably A is phenyl, pyridyl or pyrimidinyl. Even more preferably A is phenyl or pyridyl. In one embodiment A is phenyl. In another embodiment A is pyridyl.

By way of non-limiting examples for group R^{A} we refer to our applications WO 01/81350 and WO 03/022824 and references therein.

A compound of formula (IX)

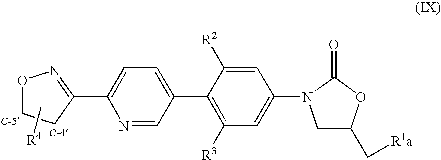

wherein:

### R¹ a is

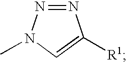

R² and R³ are independently selected from hydrogen and fluorine;
R¹ is selected from hydrogen, halogen, cyano, methyl, cyanomethyl, fluoromethyl, difluoromethyl, trifluoromethyl and —Si[(1-4C)alkyl]₃;
wherein R⁴ is either a hydroxymethyl substituent on C-4′ of the isoxazoline ring; or
R⁴ is a hydroxymethyl substituent on C-5′ of the isoxazoline ring and the stereochemistry at C-5′ of the isoxazoline ring and at C-5 of the oxazolidinone ring is selected, such that the compound of formula (I) is a single diastereomer;
or pharmaceutically-acceptable salts or pro-drugs thereof;
may be prepared by a process comprising the steps of:—

a) conversion of compound of formula (VIIa) to a compound of a formula (VIIIa)

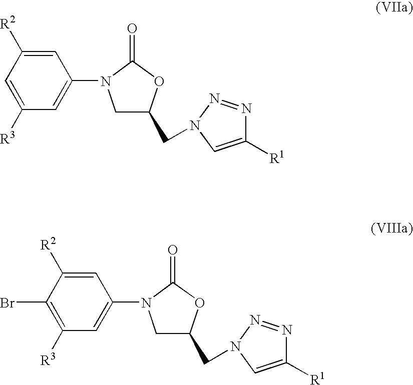

by reaction with an alkali metal bromate, a bromide and acid as described in any aspect or embodiment hereinbefore;

b) formation of a fin or boron derivative of formula (X), wherein Y is a trialkyltin or boronate acid or ester substituent;

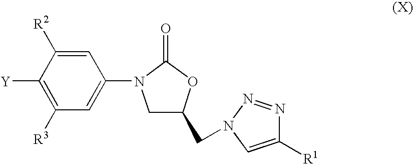

c) coupling with a compound of formula (XI) wherein R⁴ is hydroxymethyl or a protected version thereof and X is bromo or iodo;

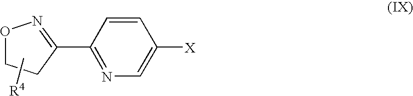

d) optionally deprotecting the hydroxymethyl substituent R⁴;

and thereafter if necessary forming a pharmaceutically-acceptable salt or pro-drug thereof.

Suitable conditions for c) (when Y is a boronic acid or ester) include coupling (X) and (XI) in the presence of a palladium (0) compound, for example tetrakis(triphenylphosphine)palladium(0). Other suitable reaction conditions are those in the presence of a palladium (II) compound, for example 1,1′-[bis(phenylphosphino)ferrocene]dichloropalladium(II) dichloromethane complex. For further information on suitable reaction conditions and catalysts for this type of coupling reaction see, for example, Kotha S. et al, Tetrahedron 2002, 58, 9633-9695.

Suitable conditions for c) (when Y is a tin derivative such as trimethyltin) include coupling (XI) and (XII) in the presence of a palladium (0) compound, for example tetrakis(triphenylphosphine)palladium(0), or triphenylphosphine and tris (dibenzylideneacetone)dipalladium (0) chloroform adduct. Further suitable reaction conditions for this process are well known in the art.

Suitable trialkyltin derivatives are any such derivative known to be useful in palladium (0) coupling reactions, such as trimethyltin.

It will be understood that by “Y is a boronic acid or ester” means Y is the group —B(OR^{A})(OR^{B}), wherein R^{A} and R^{B} are independently selected from hydrogen and a (1-4C)alkyl group (such as methyl, ethyl and isopropyl), or R^{A} and R^{B} together form a 2 or 3 carbon bridge between the two oxygen atoms attached to the boron atom to form a 5- or 6-membered ring respectively (wherein the 2 or 3 carbon bridge is optionally substituted by 1 to 4 methyl groups, for example to form a 1,1,2,2-tetramethylethylene bridge), or R^{A} and R^{B} together form a 1,2-phenyl group (thereby giving a catechol ester). This definition of boronic acid ester is similarly applicable wherever the term is applied.

For examples of protecting groups see one of the many general texts on the subject, for example, ‘Protective Groups in Organic Synthesis’ by Theodora Greene & Peter Wuts (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

The removal of any protecting groups, the formation of a pharmaceutically-acceptable salt and/or the formation of an in-vivo hydrolysable ester or other pro-drug are within the skill of an ordinary organic chemist using standard techniques. Furthermore, details on the these processes and examples of suitable salts and/or pro-drugs have been given in for example our patent application WO 03/022824.

Compounds of the formula (X) may also be prepared by a process comprising:

a) conversion of compound of formula (VIIa) to a compound of a formula (VIIIa)

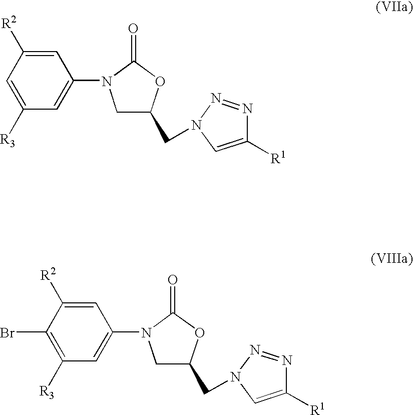

by reaction with an alkali metal bromate, a bromide and acid as described in any aspect or embodiment hereinbefore;

b′) coupling with a compound of formula (XI) wherein R⁴ is hydroxymethyl or a protected version thereof and X is a trialkyltin or boronate acid or ester substituent;

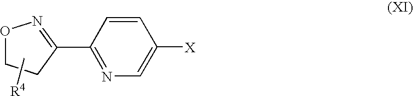

c′) optionally deprotecting the hydroxymethyl substituent R⁴;

and thereafter if necessary forming a pharmaceutically-acceptable salt or pro-drug thereof.

It will be understood that conditions for such a coupling in step b′) are directly analogous to those described hereinbefore for process step c).

The process of the invention is also useful in the preparation of a compound of formula (XII)

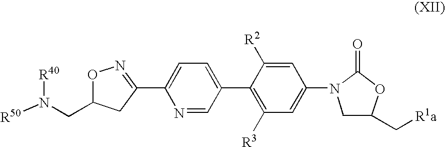

wherein:

### R¹ a is

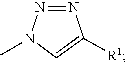

R² and R³ are independently selected from hydrogen and fluorine;
R¹ is selected from hydrogen, halogen, cyano, methyl, cyanomethyl, fluoromethyl, difluoromethyl, trifluoromethyl and —Si[(1-4C)alkyl]₃;
R⁴⁰ and R⁵⁰ are independently selected from hydrogen, allyl (optionally substituted on the carbon-carbon double bond by 1, 2 or 3 (1-4C)alkyl groups), methyl, cyanomethyl, carboxymethyl, —CH₂C(O)OR⁶⁰, —CH₂C(O)NR⁶⁰R⁷⁰, (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, azido, cyano, —C(O)OR⁶⁰, —OC(O)R⁶⁰, carboxy, —C(O)NR⁶⁰R⁷⁰, —S(O)₂R⁶⁰, —S(O)₂NR⁶⁰R⁷⁰, —NR⁶⁰R⁷⁰, —NHC(O)R⁶⁰ and —NHS(O)₂R⁶⁰], —C(O)R⁶⁰, —C(O)CH₂NR⁶⁰R⁷⁰, —C(O)OR⁶⁰, —C(O)NHR⁶⁰, —C(O)NR⁶⁰R⁷⁰ and —SO₂NHR⁶⁰;
or R⁴⁰ and R⁵⁰ together with the nitrogen to which they are attached form a 5 or 6 membered, saturated or partially unsaturated heterocyclyl ring and optionally containing 1 or 2 further heteroatoms (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl groups;
R⁶⁰ and R⁷⁰ are independently selected from hydrogen, methyl, cyclopropyl (optionally substituted with methyl), carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy);
or R⁶⁰ or R⁷⁰ may form a 4, 5 or 6 membered, carbon-linked saturated heterocyclyl ring, containing 1 or 2 heteroatoms independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom by 1 or 2 (1-4C)alkyl;
or R⁶⁰ and R⁷⁰ together with a nitrogen to which they are attached form a 4, 5 or 6 membered, saturated heterocyclyl ring, optionally containing 1 further heteroatom (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen to which R⁶⁰ and R⁷⁰ are attached is not thereby quaternised) by 1 or 2 (1-4C)alkyl groups;
provided that R⁴⁰ and R⁵⁰ are not both hydrogen.
or pharmaceutically-acceptable salts or pro-drugs thereof;
by a process comprising

a) conversion of compound of formula (VIIa) to a compound of a formula (VIIIa)

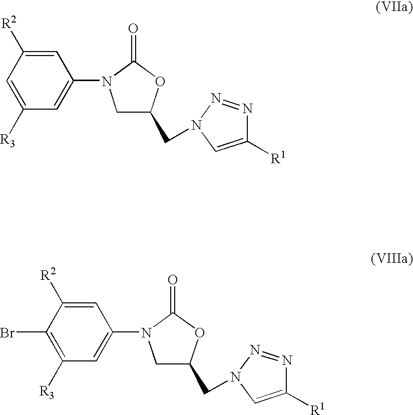

by reaction with an alkali metal bromate, a bromide and acid as described in any aspect or embodiment hereinbefore;

b) formation of a tin or boron derivative of formula (X), wherein Y is a trialkyltin or boronate acid or ester substituent;

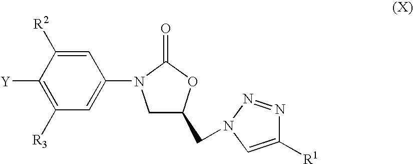

c) coupling with a compound of formula (XIa) wherein R⁴ is —CH₂NR⁴⁰R⁵⁰ or a protected version, or pre-cursor thereof and X is bromo or iodo;

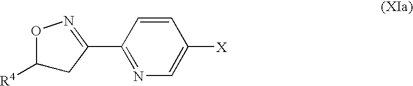

d) optionally deprotecting or converting the substituent R⁴ to give a compound of the formula (XII);

and thereafter if necessary forming a pharmaceutically-acceptable salt or pro-drug thereof.

Alternatively, a compound of the formula (XII) may be formed by a process comprising the steps of:

a) conversion of compound of formula (VIIa) to a compound of a formula (VIIIa)

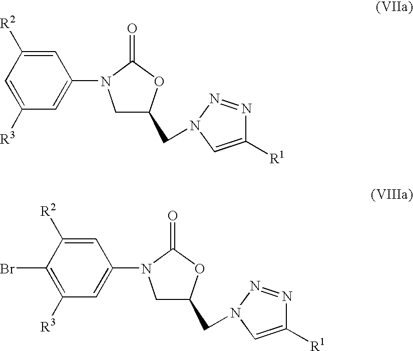

by reaction with an alkali metal bromate, a bromide and acid as described in any aspect or embodiment hereinbefore;

b′) coupling with a compound of formula (XIa) wherein R⁴ is —CH₂NR⁴⁰R⁵⁰ or a protected version, or pre-cursor thereof and X is a trialkyltin or boronate acid or ester substituent; and

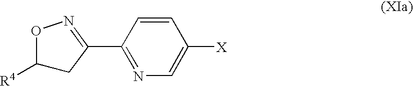

c′) optionally deprotecting or converting the substituent R⁴ to give a compound of the formula (XII);

and thereafter if necessary forming a pharmaceutically-acceptable salt or pro-drug thereof.

It will be understood that a 4, 5 or 6 membered, saturated or partially unsaturated heterocyclyl ring containing 1 or 2 heteroatoms independently selected from O, N and S (whether or not one of those heteroatoms is a linking N atom), as defined in any definition herein, does not contain any O—O, O—S or S—S bonds.

Examples of conversions of group R⁴ in step d) include for example conversion of a compound wherein R⁴ is —CH₂NHR⁴⁰ by alkylation or acylation of the remaining NH to give a compound of wherein R⁴ is CH₂NR⁴⁰R⁵⁰ and R⁵⁰ is an alkyl or acyl group.

A compound of formula (XIII)

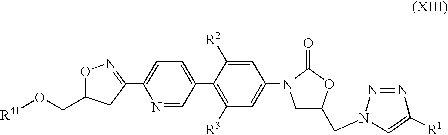

wherein:
R₂ and R₃ are independently selected from hydrogen and fluorine;
R¹ is selected from hydrogen, halogen, cyano, methyl, cyanomethyl, fluoromethyl, difluoromethyl, trifluoromethyl and —Si[(1-4C)alkyl]₃;
R⁴¹ is selected from methyl, cyanomethyl, carboxymethyl, —CH₂C(O)NR⁵¹R⁶¹ and (2-4C)alkyl [optionally substituted by 1 or 2 substituents independently selected from hydroxy, (1-4C)alkoxy, (1-4C)alkoxy(1-4C)alkoxy, hydroxy(2-4C)alkoxy, cyano, —OC(O)R⁵¹, carboxy, —C(O)NR⁵¹R⁶¹, —S(O)₂R⁵¹, —S(O)₂NR⁵¹R⁶¹, —NR⁵¹R⁶¹, —NHC(O)R⁵¹ and —NHS(O)₂R⁵¹];
R⁵¹ and R⁶¹ are independently selected from hydrogen, methyl, cyclopropyl (optionally substituted with methyl), carboxymethyl and (2-4C)alkyl (optionally substituted by 1 or 2 substituents independently selected from amino, (1-4C)alkylamino, di-(1-4C)alkylamino, carboxy, (1-4C)alkoxy and hydroxy; wherein a (1-4C)alkylamino or di-(1-4C)alkylamino group may optionally be substituted on the (1-4C)alkyl chain with carboxy);
or R⁵¹ and R⁶¹ together with a nitrogen to which they are attached form a 4, 5 or 6 membered, saturated heterocyclyl ring, optionally containing 1 further heteroatom (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen to which R⁵¹ and R⁶¹ are attached is not thereby quaternised) by 1 or 2 (1-4C)alkyl groups;
or pharmaceutically-acceptable salts or pro-drugs thereof;
may be prepared by a process comprising

a) conversion of compound of formula (VIIa) to a compound of a formula (VIIIa)

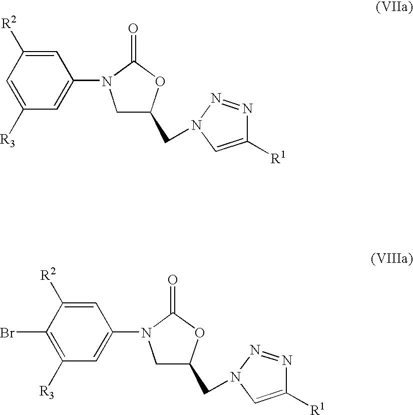

by reaction with an alkali metal bromate, a bromide and acid as described in any aspect or embodiment hereinbefore;

b) formation of a tin or boron derivative of formula (X), wherein Y is a trialkyltin or boronate acid or ester substituent;

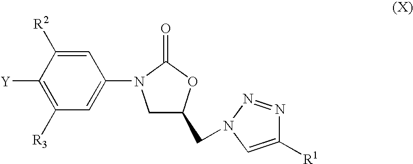

c) coupling with a compound of formula (XIa) wherein R⁴ is —CH₂OR⁴¹ or a protected version, or pre-cursor thereof and X is bromo or iodo;

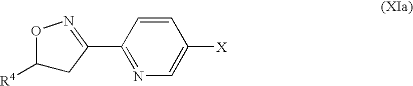

d) optionally deprotecting or converting the substituent R⁴ to give a compound of the formula (XIII);

and thereafter if necessary forming a pharmaceutically-acceptable salt or pro-drug thereof.

Alternatively a compound of the formula (XIII) may be prepared by a process comprising:

a) conversion of compound of formula (VIIa) to a compound of a formula (VIIIa)

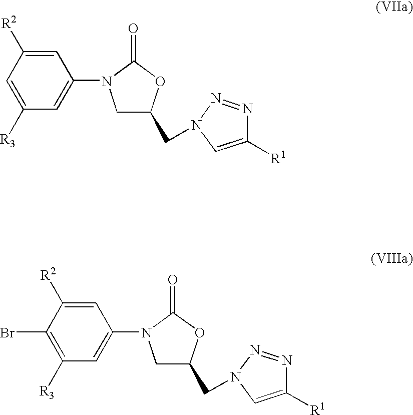

by reaction with an alkali metal bromate, a bromide and acid as described in any aspect or embodiment hereinbefore;

b′) coupling with a compound of formula (XIa) wherein R⁴ is CH₂OR⁴¹ or a protected version, or pre-cursor thereof and X is a trialkyltin or boronate acid or ester substituent; and

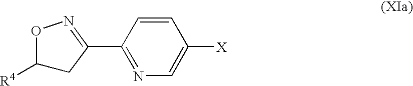

c′) optionally deprotecting or converting the substituent R⁴ to give a compound of the formula (XIII);

and thereafter if necessary forming a pharmaceutically-acceptable salt or pro-drug thereof.

The process of the invention is also useful in the synthesis of compounds of the formula (XIV):

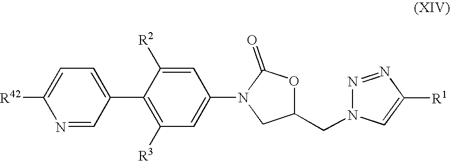

wherein R¹, R² and R³ are as hereinbefore defined;

R⁴² is (1-4C)alkyl [substituted by 1 or 2 substituents independently selected from hydroxy, (1-4C)alkoxy, (1-4C)alkoxy(1-4C)alkoxy, hydroxy(2-4C)alkoxy, —C(O)OR⁵³, —C(O)R⁵³, —OC(O)R⁵³ carboxy, —C(O)NR⁵³R⁶³, —OC(O)NR⁵³R⁶³, —S(O)₂R⁵³, —S(O)₂NR⁵³R⁶³, —NR⁵³R⁶³, —NHC(O)R⁵³ and —NHS(O)₂R⁵³; and optionally additionally substituted by cyclopropyl]; or
R⁵³ and R⁶³ are independently selected from hydrogen, methyl, cyclopropyl (optionally substituted with methyl), carboxymethyl and (2-4C)alkyl (optionally substituted by one or two substituents independently selected from amino, (1-4C)alkylamino, di-(1-4C)alkylamino, carboxy, (1-4C)alkoxy and hydroxy);
or R⁵³ and R⁶³ together with a nitrogen to which they are attached form a 4, 5 or 6 membered, saturated or partially unsaturated heterocyclyl ring, optionally containing 1 further heteroatom (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen to which R⁵³ and R⁶³ are attached is not thereby quaternised) by 1 or 2 (1-4C)alkyl groups;
or R⁵³ and R⁶³ together with a nitrogen to which they are attached form an imidazole ring, which ring is optionally substituted on an available carbon atom by 1 or 2 (1-4C)alkyl; or

### R⁴² is —C(O)R⁵⁴; or

R⁴² is selected from —C(H)═N—OR⁸⁴, —C(R⁵⁴)═N—OH and —C(R⁵⁴)═N—OR⁸⁴;
R⁵⁴ is (1-6C)alkyl (substituted with 1 or 2 substituents independently selected from hydroxy, carboxy, (1-4C)alkoxy, HET-1 and NR⁶⁴R⁷⁴);
or R⁵⁴ is (3-6C)cycloalkyl (optionally substituted with 1 substituent selected from hydroxy, carboxy, (1-4C)alkoxy and NR⁶⁴R⁷⁴);

### or R⁵⁴ is HET-1;

R⁶⁴ and R⁷⁴ are independently selected from hydrogen, methyl, cyclopropyl (optionally substituted with methyl), carboxymethyl and (2-4C)alkyl (optionally substituted by a substituent selected from amino, (1-4C)alkylamino, di-(1-4C)alkylamino, carboxy, (1-4C)alkoxy and hydroxy);
or R⁶⁴ and R⁷⁴ together with a nitrogen to which they are attached form a 4, 5 or 6 membered, saturated or partially unsaturated heterocyclyl ring, optionally containing 1 further heteroatom (in addition to the linking N atom) independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen to which R⁶⁴ and R⁷⁴ are attached is not thereby quaternised) by 1 or 2 (1-4C)alkyl groups;
or R⁶⁴ and R⁷⁴ together with a nitrogen to which they are attached form an imidazole ring, which ring is optionally substituted on an available carbon atom by 1 or 2 (1-4C)alkyl;
R⁸⁴ is (1-6C)alkyl (optionally substituted with 1 or 2 substituents independently selected from hydroxy, carboxy, (1-4C)alkoxy and NR⁶⁴R⁷⁴);
HET-1 is a 5 or 6 membered saturated or partially unsaturated heterocyclyl ring, containing 1 or 2 heteroatoms independently selected from O, N and S, wherein a —CH₂— group may optionally be replaced by a —C(O)— and wherein a sulphur atom in the ring may optionally be oxidised to a S(O) or S(O)₂ group; which ring is optionally substituted on an available carbon or nitrogen atom (providing the nitrogen is not thereby quaternised) by 1 or 2 (1-4C)alkyl.

It will be appreciated that in this last aspect, the compound of formula (VIIIa) will be coupled with a compound of formula (XV), wherein X is halo, such as bromo or iodo:

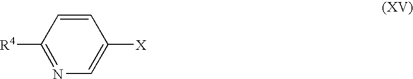

It will be appreciated that a compound of the formula (VIIa), (VIIIa), (X), (XII), (XIII) or (IX) wherein R¹ is —Si[(1-4C)alkyl]₃ may be converted to another compound of the formula (VIIa), (VIIIa), (X), (XII), (XIII) or (IX) respectively wherein R¹ is selected from hydrogen, halogen, cyano, methyl, cyanomethyl, fluoromethyl, difluoromethyl and trifluoromethyl, as a separate process step at any appropriate point.

Compounds of the formula (XI) may be derived from an oxime substituted pyridine derivative as shown below, wherein X is Br or I. The oxime derivative itself may be derived from simple halo-pyridine derivatives via aldehydro-halopyridines. The chiral centre on the isoxazole ring may be introduced by any means known in the art, for example by resolution of an ester group, for instance using an enzyme such as a lipase to achieve selectivity. This process is illustrated below for a butyrate ester, however it will be appreciated that other alkyl or alkenyl esters may be used, and that resolution and hydrolysis may be achieved in a single step by enzyme catalysed selective ester hydrolysis. It will be appreciated that X in formula (XI) as shown in the scheme below may be the same throughout the assembly of the 2 ring system, or may be altered at an appropriate point prior to coupling with the compound of formula (X):

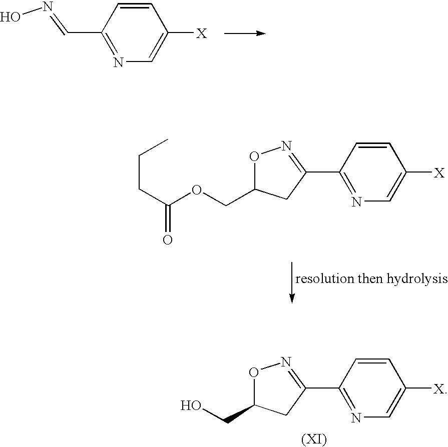

The hydroxymethyl substituent in (XI) above may then be elaborated by using standard chemistry to form a compound of the formula (XIa) wherein R⁴ is —CH₂OR⁴¹, or converted using standard chemistry to a compound of formula (XIa) wherein R⁴ is —CH₂NR⁴⁰R⁵⁰.

Compounds of the formula (XV) may be made for example by functionalisation of a di-halopyridine derivative, for example by alkylation using a Grignard reagent.

There follow particular and suitable values for certain substituents and groups referred to in this specification. These values may be used where appropriate with any of the definitions and embodiments disclosed hereinbefore, or hereinafter. For the avoidance of doubt each stated species represents a particular and independent aspect of this invention.

Examples of (1-4C)alkyl include methyl, ethyl, propyl, isopropyl and t-butyl; examples of (2-4C)alkyl include ethyl, propyl, isopropyl and t-butyl; examples of (1-6C)alkyl include methyl, ethyl, propyl, isopropyl, t-butyl, pentyl and hexyl; examples of hydroxy(1-4C)alkyl include hydroxymethyl, 1-hydroxyethyl, 2-hydroxyethyl and 3-hydroxypropyl; examples of hydroxy(2-4C)alkyl include 1-hydroxyethyl, 2-hydroxyethyl, 2-hydroxypropyl, 3-hydroxypropyl, 1-hydroxyisopropyl and 2-hydroxyisopropyl; examples of (1-4C)alkoxycarbonyl include methoxycarbonyl, ethoxycarbonyl and propoxycarbonyl; examples of (2-4C)alkenyl include allyl and vinyl; examples of (2-4C)alkynyl include ethynyl and 2-propynyl; examples of (1-4C)alkanoyl include formyl, acetyl and propionyl; examples of (1-4C)alkoxy include methoxy, ethoxy and propoxy; examples of (1-6C)alkoxy and (1-10C)alkoxy include methoxy, ethoxy, propoxy and pentoxy; examples of (1-4C)alkylthio include methylthio and ethylthio; examples of (1-4C)alkylamino include methylamino, ethylamino and propylamino; examples of di-((1-4C)alkyl)amino include dimethylamino, N-ethyl-N-methylamino, diethylamino, N-methyl-N-propylamino and dipropylamino; examples of halo groups include fluoro, chloro and bromo; examples of (1-4C)alkoxy-(1-4C)alkoxy and (1-6C)alkoxy-(1-6C)alkoxy include methoxymethoxy, 2-methoxyethoxy, 2-ethoxyethoxy and 3-methoxypropoxy; examples of (1-4C)alkanoylamino and (1-6C)alkanoylamino include formamido, acetamido and propionylamino; examples of (1-4C)alkylS(O)_{q}— wherein q is 0, 1 or 2 include methylthio, ethylthio, methylsulfinyl, ethylsulfinyl, methylsulfonyl and ethylsulfonyl; examples of hydroxy-(2-4C)alkoxy include 2-hydroxyethoxy and 3-hydroxypropoxy; examples of (1-6C)alkoxy-(1-6C)alkyl and (1-4C)alkoxy(1-4C)alkyl include methoxymethyl, ethoxymethyl and propoxyethyl; examples of (1-4C)alkylcarbamoyl include methylcarbamoyl and ethylcarbamoyl; examples of di((1-4C)alkyl)carbamoyl include di(methyl)carbamoyl and di(ethyl)carbamoyl; examples of halo groups include fluoro, chloro and bromo; examples of halo(1-4C)alkyl include, halomethyl, 1-haloethyl, 2-haloethyl, and 3-halopropyl; examples of dihalo(1-4C)alkyl include difluoromethyl and dichloromethyl; examples of trihalo(1-4C)alkyl include trifluoromethyl; examples of amino(1-4C)alkyl include aminomethyl, 1-aminoethyl, 2-aminoethyl and 3-aminopropyl; examples of cyano(1-4C)alkyl include cyanomethyl, 1-cyanoethyl, 2-cyanoethyl and 3-cyanopropyl; examples of (1-4C)alkanoyloxy include acetoxy, propanoyloxy; examples of (1-6C)alkanoyloxy include acetoxy, propanoyloxy and tert-butanoyloxy; examples of (1-4C)alkylaminocarbonyl include methylaminocarbonyl and ethylaminocarbonyl; examples of di((1-4C)alkyl)aminocarbonyl include dimethylaminocarbonyl and diethylaminocarbonyl.

In the formation of a compound of formula (IX), (XII), (XIII), or (XIV) described above, steps b) and c) may be carried out without isolation of the intervening tin or boron compound of formula (X), as illustrated by Example 2 hereinafter.

A further aspect of the invention comprises the use of a compound of formula (VIII) in a process to make a compound of formula (IX), (XII), (XIII) or (XIV).

### EXAMPLES

The invention is now illustrated but not limited by the following Examples in which unless otherwise stated:—
(i) evaporations were carried out by rotary evaporation in vacuo and work-up procedures were carried out after removal of residual solids by filtration;
(ii) operations were carried out at ambient temperature, that is typically in the range 18-26° C. and without exclusion of air unless otherwise stated, or unless the skilled person would otherwise work under an inert atmosphere;
(iii) column chromatography (by the flash procedure) was used to purify compounds and was performed on Merck Kieselgel silica (Art. 9385) unless otherwise stated;
(iv) yields are given for illustration only and are not necessarily the maximum attainable;
(v) the structure of the end-products of the invention were generally confirmed by NMR and mass spectral techniques [proton magnetic resonance spectra were generally determined in DMSO-d₆ unless otherwise stated using a Varian Gemini 2000 spectrometer operating at a field strength of 300 MHz, or a Bruker AM250 spectrometer operating at a field strength of 250 MHz, or a Bruker DPX400 spectrometer operating at a field strength of 400 MHz; chemical shifts are reported in parts per million downfield from tetramethysilane as an internal standard (δ scale) and peak multiplicities are shown thus: s, singlet; d, doublet; AB or dd, doublet of doublets; dt, doublet of triplets; dm, doublet of multiplets; t, triplet, m, multiplet; br, broad. Time of flight (TOF) mass spectral data were obtained using a Micromass LCT mass spectrometer; fast-atom bombardment (FAB) mass spectral data were generally obtained using a Platform spectrometer (supplied by Micromass) run in electrospray and, where appropriate, either positive ion data or negative ion data were collected]; optical rotations were determined at 589 nm at 20° C. for 0.1M solutions in methanol using a Perkin Elmer Polarimeter 341;
(vi) each intermediate was purified to the standard required for the subsequent stage and was characterised in sufficient detail to confirm that the assigned structure was correct; purity was assessed by HPLC, TLC, or NMR and identity was determined by infra-red spectroscopy (IR), mass spectroscopy or NMR spectroscopy as appropriate;
(vii) in which the following abbreviations may be used:—

DMF is N,N-dimethylformamide; DMA is N,N-dimethylacetamide; TLC is thin layer chromatography; HPLC is high pressure liquid chromatography; MPLC is medium pressure liquid chromatography; DMSO is dimethylsulfoxide; CDCl₃ is deuterated chloroform; MS is mass spectroscopy; TOF is time of flight; ESP is electrospray; EI is electron impact; CI is chemical ionisation; APCI is atmospheric pressure chemical ionisation; EtOAc is ethyl acetate; MeOH is methanol; phosphoryl is (HO)₂—P(O)—O—; phosphiryl is (HO)₂—P—O—; Bleach is “Clorox” 6.15% sodium hypochlorite; EDAC is 1-[3-(dimethylamino)propyl]-3-ethylcarbodiimide; THF is tetrahydrofuran; TFA is trifluoroacetic acid; RT is room temperature; cf.=compare
(viii) temperatures are quoted as ° C.
(ix) MP carbonate resin is a solid phase resin for use in acid Scavenging, available from Argonaut Technologies, chemical structure is PS—CH₂N(CH₂CH₃)₃⁺(CO₃²⁻)_{0.5}

### Example 1

### (5R)-3-(4-Bromo-3-fluorophenyl)-5-(1H-1,2,3-triazol-1-ylmethyl)-1,3-oxazolidin-2-one

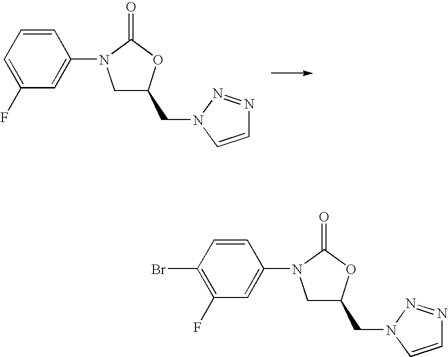

To a stirred solution of (5R)-3-(3-fluorophenyl)-5-(1H-1,2,3-triazol-1-ylmethyl)-1,3-oxazolidin-2-one (Intermediate 3) (2.0 g, 7.63 mmol) in acetic acid (6.0 mL) and water (4.0 mL) at 14° C. was added 48% w/w aqueous hydrobromic acid solution (1.0 mL) followed by dropwise addition of a solution of sodium bromate (580 mg, 3.81 mmol) in water (3.0 mL) over 2.5 hours. After stirring the mixture at 26° C. for a further 1.5 hours, a solution of sodium metabisulfite (0.435 g, 2.29 mmol) in water (1.0 mL) was added over about 10 minutes. The mixture was heated to 83° C. to give a clear solution, which was then cooled to 5° C. The resultant slurry was stirred for a further 1 hour before the solid product was isolated by filtration, washed thrice with water (4.0 mL), and dried in vacuo at up to 50° C. to yield the title compound (2.4 g).

MS (TOF): 341.0043 (M+1) for C₁₂H₁₀N₄O₂FBr⁷⁹; calc., 341.0049

¹H NMR (400 MHz, DMSO-d₆) δ: 3.9 (dd, 1H), 4.2 (t, 1H), 4.8 (d, 2H), 5.2 (m, 1H), 7.3 (ddd, 1H), 7.6 (dd, 1H), 7.7 (dd, 1H), 7.8 (d, 1H), 8.2 (d, 1H).

The intermediates for Example 1 were prepared as follows:

### Intermediate 1: (5R)-5-(Hydroxymethyl)-3-(3-fluorophenyl)-1,3-oxazolidin-2-one

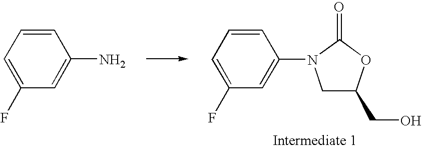

A stirred solution of 3-fluoroaniline (45.68 g, 0.41 mol) in toluene (447 mL) was heated to 30° C. and treated with pyridine (39.1 mL, 0.48 mol). iso-Butyl chloroformate (62 mL, 0.48 mol) was added over about 0.5 hour and the mixture was stirred for about 3 hours at 30° C. Water (134 mL) was added and the mixture was stirred at 30° C. before the layers were separated. The organic layer was further washed with water (134 mL) and then distilled under reduced pressure until about 210 mL of distillate was collected.

The stirred residue was diluted with toluene (376 mL) and tetrahydrofuran (376 mL) and then cooled to −10° C. A solution of n-butyl lithium in toluene (24.5% w/w, 3.3M, 113.6 mL, 0.377 mol) was added over 50 minutes, followed by addition of R-glycidyl butyrate (57.3 mL, 0.40 mol). The mixture was warmed to 40° C. over about 3 hours and this temperature was maintained for a further 1.5 hour before methanol (192.6 mL) was added. The resultant clear solution was added to a mixture of acetic acid (24.5 mL) and water (170 mL) and the layers were allowed to separate. The organic extracts were stirred overnight with water (250 mL), separated, and washed again with water (100 mL), before distilling under reduced pressure to leave about 400 mL of concentrate, which was cooled to 0° C. over a period of about 16 hours and stirred for a further 4 hours at this temperature to give a suspension. The solid was isolated by filtration, washed with toluene (50 mL) and dried in vacuo at up to 40° C. to give the title compound (61.97 g).

MS (TOF): 212.0729 (M+1) for C₁₀H₁₀NO₃F; calc., 212.0723

¹H NMR (400 MHz, DMSO-d₆) δ: 3.6 (ddd, 1H), 3.7 (ddd, 1H), 3.8 (dd, 1H), 4.1 (t, 1H), 4.7 (m, 1H), 5.2 (t, 1H), 6.9 (m, 1H), 7.3 (ddd, 1H), 7.4 (td, 1H), 7.5 (dt, 1H).

### Intermediate 2: [(5R)-3-(3-Fluorophenyl)-2-oxo-1,3-oxazolidin-5-yl]methyl Methanesulfonate

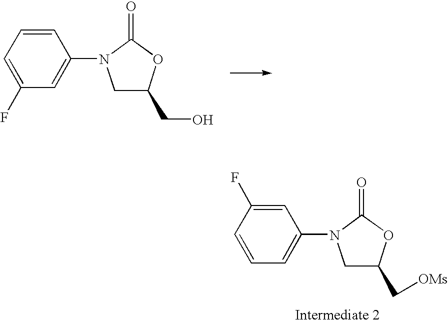

A stirred suspension of (5R)-5-(Hydroxymethyl)-3-(3-fluorophenyl)-1,3-oxazolidin-2-one (20.0 g, 0.095 mol) in toluene (300 mL) was heated to 85° C. to give a solution, and triethylamine (20.17 mL, 0.142 mol) was added. The solution was cooled to 68° C., and methanesulfonyl chloride (9.2 mL, 0.118 mol) was added over 10 minutes. Water (200 mL) was added to the mixture and the mixture was stirred at 75° C. for 0.5 hour. The mixture was then cooled to 15° C. over about 2 hours giving a slurry of the product. The solid was isolated by filtration, washed with water (200 mL), and dried to give the title compound (27.3 g).

MS (TOF): 290.0506 (M+1) for C₁₁H₁₂NO₅FS; calc., 290.0498

¹H NMR (400 MHz, DMSO-d₆) δ: 3.3 (s, 3H), 3.8 (dd, 1H), 4.2 (t, 1H), 4.5 (dd, 1H), 4.5 (dd, 1H), 5.0 (m, 1H), 7.0 (m, 1H), 7.3 (ddd, 1H), 7.4 (td, 1H), 7.5 (dt, 1H).

### Intermediate 3: (5R)-3-(3-Fluorophenyl)-5 (1H-1,2,3-triazol-1-ylmethyl)-1,3-oxazolidin-2-one

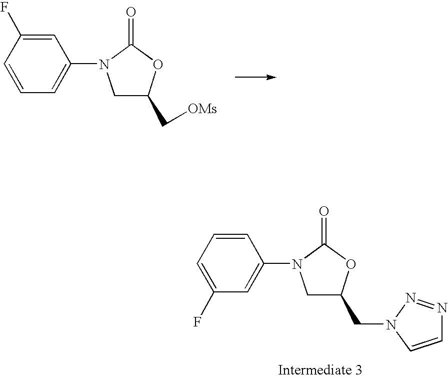

A stirred suspension of [(5R)-3-(3-fluorophenyl)-2-oxo-1,3-oxazolidin-5-yl]methyl methanesulfonate (25 g, 0.086 mol) and sodium azide (6.18 g, 0.095 mol) in N-methyl-pyrrolidinone (250 mL) was heated to 95° C. for about 3 hours and then cooled to 20° C. The mixture was filtered and to the filtrates was added (trimethylsilyl)acetylene (24.9 mL, 0.172 mol). The mixture was heated to 135° C. and this temperature was maintained for 5 hours. During this time, two further portions of (trimethylsilyl)acetylene (2.0 mL) were added after 2.5 hours and 3.5 hours. Water (10 mL) was added and heating was continued for 0.5 hour. The reaction mixture was cooled to 125° C., water (10 mL) and (trimethylsilyl)acetylene (2.0 mL) were added before heating at 125° C. was continued for a further 2 hours. The reaction was cooled to 25° C. and evaporated under reduced pressure to give an oily residue. The residue was diluted with acetic acid (95 mL) and water (25 mL) and heated with stirring to 91° C. for 5.5 hours, before cooling to 30° C. The mixture was heated to 61° C. and water (300 mL) was added over 1 hour, before cooling to 15° C. over abut 2.5 hours, to give a suspension of the product. The crude product was isolated by filtration, washed thrice with water (50 mL) and recrystallised from a mixture of acetic acid (95 mL) and water (245 mL). The product was isolated by filtration, washed thrice with water (50 mL), and dried in vacuo at up to 40° C. to give the title compound (5.53 g).

MS (TOF): 263.0939 (M+1) for C₁₂H₁₁N₄O₂F; calc., 263.0944

¹H NMR (400 MHz, DMSO-d₆) δ: 3.9 (dd, 1H), 4.2 (t, 1H), 4.8 (d, 2H), 5.2 (m, 1H), 7.0 (m, 1H), 7.3 (ddd, 1H), 7.4 (m, 2H), 7.8 (d, 1H), 8.2 (d, 1H).

### Example 2

### Conversion to (5R)-3-[3-Fluoro-4-[((5S)-5-hydroxymethyl-4,5-dihydroisoxazol-3-yl)-3-pyridinyl]phenyl]-5-(1H-1,2,3-triazol-1-ylmethyl)oxazolidin-2-one

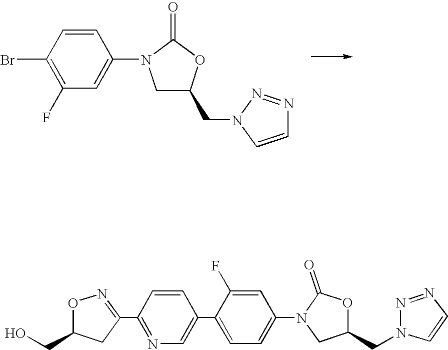

To a stirred mixture of (5R)-3-(4-Bromo-3-fluorophenyl)-5-(1H-1,2,3-triazol-1-ylmethyl)-1,3-oxazolidin-2-one (Example 1) (60.0 g, 0.175 mol), potassium acetate (49.41 g, 0.498 mol) and bis(pinacolato)diboron (51.11 g, 0.199 mol) in 1,4-dioxan (750 mL) was added 1,1′-[bis(diphenylphosphino)ferrocene]dichloropalladium(II) dichloromethane complex (2.74 g, 0.0033 mol), and the resultant mixture was heated to 82° C. This temperature was maintained for 21 hours and the mixture was then cooled to 25° C. before filtration. The solids were washed with 1,4-dioxan (180 mL) and to the combined filtrates and washings were added [(5S)-3-(5-Bromopyridin-2-yl)-4,5-dihydroisoxazol-5-yl]methanol (Intermediate 5, 42.7 g, 0.166 mol) and 1,1′-[bis(diphenylphosphino)ferrocene]dichloropalladium(II) dichloromethane complex (0.68 g, 0.00083 mol), followed by a solution of potassium carbonate (45.9 g, 0.332 mol) in water (300 mL). The resulting biphasic mixture was stirred and heated at 80° C. for 1.5 hours. After cooling to 50° C. the lower, aqueous layer was removed, and the organic layer was further cooled to 30° C., giving a suspension of the product, which was isolate by filtration, washed with a mixture of 1,4-dioxan (180 mL) and water (60 mL), water (60 mL), and methanol (120 mL), and then dried in vacuo at up to 40° C. to give the title compound (56.23 g).

MS (TOF): 439.1535 (M+1) for C₂₁H₁₉N₆O₄F; calc., 439.1530

¹H NMR (400 MHz, DMSO-d₆) δ: 3.3 (dd, 1H), 3.5 (dd, 1H), 3.6 (m, 2H), 4.0 (dd, 1H), 4.3 (t, 1H), 4.8 (m, 1H), 4.9 (d, 2H), 5.0 (t, 1H), 5.2 (m, 1H), 7.4 (dd, 1H), 7.6 (dd, 1H), 7.7 (t, 1H), 7.8 (d, 1H), 8.0 (broad d, 1H), 8.1 (broad d, 1H), 8.2 (d, 1H), 8.8 (broad s, 1H).

### Example 3

### Stepwise Conversion to (5R)-3-[3-Fluoro-4-[((5S)-5-hydroxymethyl-4,5-dihydroisoxazol-3-yl)-3-pyridinyl]phenyl]-5-(1H-1,2,3-triazol-1-ylmethyl)oxazolidin-2-one

### (i) (5R)-3-[3-Fluoro-4-(4,4,5,5-tetramethyl-1,3,2-dioxaborolan-2-yl)phenyl]-5-(1H-1,2,3-triazol-1-ylmethyl)-1,3-oxazolidin-2-one

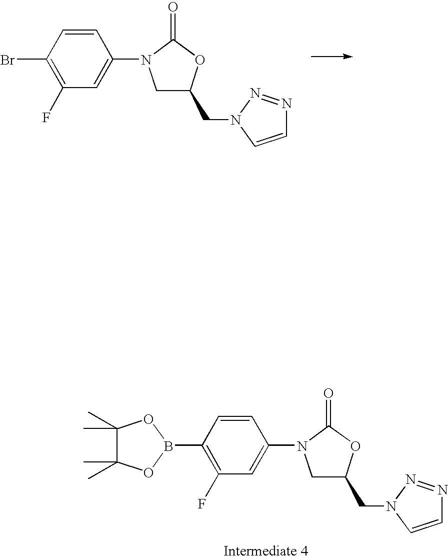

Charcoal (6.0 g) was added to a stirred solution of (5R)-3-(4-Bromo-3-fluorophenyl)-5-(1H-1,2,3-triazol-1-ylmethyl)-1,3-oxazolidin-2-one (Example 1) (60.0 g, 0.175 mol) in 1,4-dioxan (600 mL) and the mixture was heated to 60° C. for 0.5 hour. The mixture was cooled to 30° C. and filtered, the filter cake was washed with further 1,4-dioxan (150 mL). Potassium acetate (65.88 g, 0.66 mol), bis(pinacolato)diboron (51.11 g, 0.199 mol) and 1,1′-[bis(diphenylphosphino)ferrocene]dichloropalladium(II) dichloromethane complex (2.74 g, 0.0033 mol) were added to the combined filtrates and washings, and the resultant mixture was heated to 82° C. After 18 hours, the reaction mixture was cooled to 25° C. and filtered and the filtered solids were washed with a further portion of 1,4-dioxan (180 mL). The combined filtrates and washings were evaporated to dryness under reduced pressure. The residue was dissolved in n-butyl acetate (400 mL) by heating to 110° C. and cooled to 40° C. before charcoal (6.0 g) was added. The mixture was heated to 90° C. and whilst still hot, solids were removed by filtration. The filtrates were cooled to ambient temperature, the solid product was isolated by filtration, washed with n-butyl acetate (200 mL) and iso-hexane (400 mL), and air dried to give the title compound (35.77 g) (Intermediate 4)

MS (TOF): 389.1800 (M+1) for C₁₈H₂₂B¹¹N₄O₄F; calc., 389.1796

¹H NMR (400 MHz, DMSO-d₆) δ: 1.3 (s, 12H), 3.9 (dd, 1H), 4.2 (t, 1H), 4.8 (d, 2H), 5.2 (ddd, 1H), 7.3 (dd, 1H), 7.4 (dd, 1H), 7.6 (dd, 1H), 7.8 (d, 1H), 8.2 (d, 1H).

### (ii)(5R)-3-(3-Fluoro-4-{6-[(5S)-5-(hydroxymethyl)-4,5-dihydroisoxazol-3-yl]pyridin-3-yl}phenyl)-5-(1H-1,2,3-triazol-1-ylmethyl)-1,3-oxazolidin-2-one

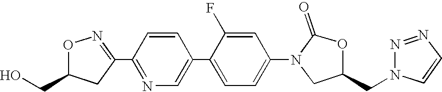

[(5S)-3-(5-Bromopyridin-2-yl)-4,5-dihydroisoxazol-5-yl]methanol (Intermediate 5, 0.277 g, 1.08 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (Intermediate 4)(0.35 g, 0.9 mmol), potassium carbonate (0.622 g, 4.5 mmol), and tetrakis(triphenylphosphino)palladium(0) (0.1 g, 0.09 mmol) were combined and suspended in DMF (7 ml) and water (1 ml). The mixture was heated at 75° C. for 2 hours, then was poured into cold water (30 ml). The solids formed were collected, rinsed with water and washed with dichloromethane (2×10 ml), the solids were then dissolved in warm trifluoroethanol (2 ml), and further purified by column chromatography, eluting with 8% methanol in dichloromethane to give the title compound as a white solid (0.193 g).

MS (ESP): 439.22 (M+1) for C₂₁H₁₉FN₆O₄

NMR (300 Mz) (DMSO-d₆) δ: 3.36-3.58 (m, 4H); 3.95 (dd, 1H); 4.29 (t, 1H); 4.78 (m, 1H); 4.86 (d, 2H); 5.02 (t, 1H); 5.18 (m, 1H); 7.41 (dd, 1H); 7.58 (dd, 1H); 7.69 (t, 1H); 7.77 (s, 1H); 7.98 (d, 1H); 8.05 (dd, 1H); 8.18 (s, 1H); 8.78 (s, 1H).

The intermediates for Examples 2 and 3 were prepared as follows:—

### Intermediate 5: [(5S)-3-(5-Bromopyridin-2-yl)-4,5-dihydroisoxazol-5-yl]methanol

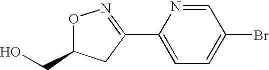

[(5S)-3-(5-Bromopyridin-2-yl)-4,5-dihydroisoxazol-5-yl]methyl butyrate (Intermediate 6, 16.88 g, 0.051 mol) was dissolved in methanol (110 ml). 50% Aqueous sodium hydroxide (3.6 ml, 0.068 mol) was added. The solution was stirred at RT for 15 minutes, 1M HCl (75 ml) was added, followed by concentration in vacuo to ˜100 ml total volume. Water (˜50 ml) was added, and the white precipitate was collected and rinsed with water. The filtrate was extracted twice with ethyl acetate, the organic layers were pooled, dried over sodium sulfate and evaporated. The solid residue was collected and rinsed with 10:1 hexane:ethyl acetate, then combined with the initial precipitate before drying in vacuo to give the title compound as a white crystalline solid, 12.3 g (93%). Chiral HPLC analysis indicated <0.5% of the (−) isomer was present. [α]_{D}=+139 (c=0.01 g/ml in methanol).

### Intermediate 6: (5S)-3-(5-Bromopyridin-2-yl)-4,5-dihydroisoxazol-5-yl]methyl Butyrate

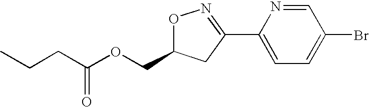

(+) Isomer assigned as (5S) based on comparison with Chem. Lett. 1993 p. 1847.

Racemic [3-(5-bromopyridin-2-yl)-4,5-dihydroisoxazol-5-yl]methyl butyrate (Intermediate 7, 80 g, 0.244 mol) was dissolved in acetone (4 L), and 0.1 M potassium phosphate buffer (pH˜7) (4 L) was added with vigorous stirring to give a clear yellow solution. PS-lipase (1.45 g, Sigma cat no L-9156) was added and the mixture was gently stirred at ambient temp. for 42 hrs. The solution was divided into 3 equal volumes of ˜2.6 L and each was extracted with dichloromethane (2×1 L), the pooled organic phases were dried over sodium sulfate and evaporated. The unreacted [(5S)-3-(5-bromopyridin-2-yl)-4,5-dihydroisoxazol-5-yl]methyl butyrate was isolated via flash column chromatography (9:1 hexane:ethyl acetate) as a clear yellow oil, 36.4 g (45.5%).

### Intermediate 7: [3-(5-Bromopyridin-2-yl)-4,5-dihydroisoxazol-5-yl]methyl Butyrate

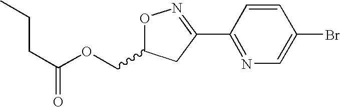

5-Bromo-N-hydroxypyridine-2-carboximidoyl chloride (Intermediate 8, 46 g, 195.7 mmol) was added to EtOAc (200 ml) followed by addition of allyl butyrate (145 ml, 1020.4 mmol) and the solution was cooled to 0° C. Triethylamine (30 ml, 215.8 mmol) in EtOAc (100 ml) was then added dropwise over 1 hour. The reaction was then allowed to stir for 1 hour at 0° C. and then BtOAc (1 L) was added. The precipitate was removed by vacuum filtration and the filtrate was concentrated in vacuo to yield the product (65 g).

¹H-NMR (DMSO-d₆) δ: 0.81 (t, 3H); 1.43 (m, 2H); 2.24 (t, 2H); 3.21 (dd, 1H); 3.54 (dd, 1H); 4.13 (dd, 1H); 4.23 (dd, 1H); 5.01 (m, 1H); 7.85 (dd, 1H); 8.12 (dd, 1H); 8.81 (d, 1H).

### Intermediate 8: 5-Bromo-N-hydroxypyridine-2-carboximidoyl Chloride

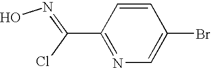

5-Bromopyridine-2-carbaldehyde oxime (Intermediate 9, 49.5 g, 246.3 mmol) was dissolved in DMF (150 ml) followed by addition of N-chlorosuccinimide (39.5 g, 295.5 mmol). HCl gas was then bubbled in the solution for 20 seconds to initiate the reaction, which was then allowed to stir for 1 hr. The reaction was poured into distilled water (1 L) and the precipitate was collected by vacuum filtration. The filter cake was washed with distilled water (2×500 ml) and then dried overnight in a vacuum oven at 60° C. (−30 inches Hg) to yield the product as a white powder (55 g).

¹H-NMR (300 Mz)(CDCl₃) δ: 7.73 (d, 1H); 8.09 (d, 1H); 8.73 (s, 1H); 12.74 (s, 1H).

### NOTE: Lachrymator.

### Intermediate 9: 5-Bromopyridine-2-carbaldehyde Oxime

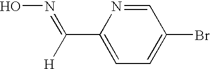

5-Bromo-pyridine-2-carbaldehyde (X. Wang et al, Tetrahedron Letters 41 (2000), 4335-4338) (60 g, 322 mmol) was added to methanol (700 ml) and then water was added (700 ml) followed by addition of hydroxylamine hydrochloride (28 g, 403 mmol). Sodium carbonate (20.5 g, 193.2 mmol) in water (200 ml) was added and the reaction was stirred for 30 minutes. Water (500 ml) was then added and the precipitate was filtered and washed with water (2×300 ml) to give the desired product (60 g).

NMR (DMSO-d₆) δ: 7.75 (d, 1H); 8.09 (t, 2H), 8.72 (s, 1H); 11.84 (s, 1H).

### Example 4

### Stepwise Conversion to (5R)-3-(3-Fluoro-4-{6-[(5R)-5-(morpholin-4-ylmethyl)-4,5-dihydroisoxazol-3-yl]pyridin-3-yl}phenyl)-5-(1H-1,2,3-triazol-1-ylmethyl)-1,3-oxazolidin-2-one

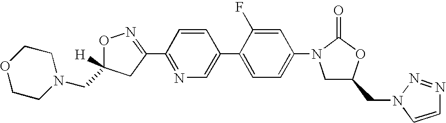

4-{[(5R)-3-(5-Bromopyridin-2-yl)-4,5-dihydroisoxazol-5-yl]methyl}morpholine (Intermediate 12, 320 mg, 0.98 mmol), (5R)-3-[3-fluoro-4-(4,4,5,5-tetramethyl-1,3,2-dioxaborolan-2-yl)phenyl]-5-(1H-1,2,3-triazol-1-ylmethyl)-1,3-oxazolidin-2-one (400 mg, 1.03 mmol), potassium carbonate (Intermediate 4, 450 mg, 3.26 mmol), and tetrakis(triphenylphosphino)palladium(0) (120 mg, 0.10 mmol) were suspended in DMF (5 ml) and water (0.5 ml). The mixture was heated at 80° C. for 60 minutes, allowed to cool, and filtered. The solids were rinsed with acetonitrile and the combined filtrate was adsorbed on silica gel. The adsorbed material was purified by column chromatography (silica gel, 1 to 10% methanol in dichloromethane). The off-white solid thus obtained (430 mg) was dissolved in hot dioxane (30 ml) and treated with HCl (4M solution in dioxane, 0.25 ml, 1 mmol) to give a suspension, which was diluted with diethyl ether (50 ml) followed by filtration and rinsing with diethyl ether. The hydrochloride salt of the title compound was thus obtained as an off-white solid (400 mg): melting point: 239-245° C.

MS (electrospray): 508 (M+1) for C₂₅H₂₆FN₇O₄

¹H-NMR (400 MHz, DMSO-d₆) δ: 3.18 (bm, 2H); 3.37 (dd, 1H); 3.49 (bm, 3H); 3.77 (m, 4H); 3.96 (m, 3H); 4.30 (t, 1H); 4.86 (d, 2H); 5.19 (m, 1H); 5.32 (m, 1H); 7.42 (dd, 1H); 7.59 (dd, 1H); 7.69 (t, 1H); 7.76 (s, 1H); 8.02 (d, 1H); 8.09 (d, 1H); 8.18 (s, 1H); 8.81 (s, 1H); 10.55 (bs, 1H).

The intermediates for Example 4 were prepared as follows:—

### Intermediate 10: [(5R)-3-(5-bromopyridin-2-yl)-4,5-dihydroisoxazol-5-yl]methanol

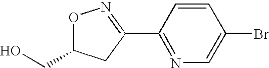

(R,S)-[3-(5-Bromopyridin-2-yl)-4,5-dihydroisoxazol-5-yl]methanol (prepared by hydrolysis of Intermediate 7, 3.1 g) was dissolved in hot methanol (25 ml), it was then separated by chiral column (Chiral Pak AS) eluting with 30% isopropanol in hexanes. The title compound [(−) isomer, 1.5 g)] which eluted first from the column was collected along with the (+) isomer (second peak, 1.18 g). Chiral HPLC analysis indicated <2% of the (+) isomer was present. [α]_{D}=−125° (c=0.0076 g/ml in methanol).

### Intermediate 11: 5-Bromo-2-[(5R)-5-(chloromethyl)-4,5-dihydroisoxazol-3-yl]pyridine

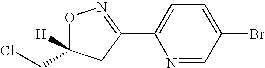

[(SR)-3-(5-Bromopyridin-2-yl)-4,5-dihydroisoxazol-5-yl]methanol (Intermediate 10, 0.274 g, 1.06 mmol) was dissolved in dichloromethane (5 ml). Triphenylphosphine (0.8 g, 3.05 mmol) and carbon tetrachloride (0.6 ml, 6.2 mmol) were added and the mixture was stirred at room temperature for 2 hours. Methanol (0.5 ml) was added, and the solution was concentrated and purified by flash chromatography (silica gel, 5 to 20% ethyl acetate in hexane) to yield the title compound as a white solid (280 mg).

¹H-NMR (300 MHz, CDCl₃) δ: 3.42-3.73 (m, 4H); 4.98-5.08 (m, 1H); 7.84 (dd, 1H); 7.90 (d, 1H); 8.65 (d, 1H).

### Intermediate 12: 4-{[(5R)-3-(5-Bromopyridin-2-yl)-4,5-dihydroisoxazol-5-yl]methyl}morpholine

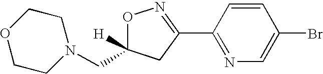

5-Bromo-2-[(5R)-5-(chloromethyl)-4,5-dihydroisoxazol-3-yl]pyridine (Intermediate 11, 0.276 g, 1.0 mmol), morpholine (0.9 ml, 10.3 mmol), tetrabutyl ammonium iodide (2 mg, catalytic amount) and DMSO (0.9 ml) were combined and warmed to 115° C. for 4 hours. The solution was diluted with water and extracted twice with ethyl acetate. The pooled organic layers were dried over sodium sulfate and evaporated to give crude title compound as a waxy yellow solid (320 mg).

MS (electrospray): 327 (M+1) for C₁₃H₁₆BrN₃O₂

What is claimed is:

1. A process for forming a compound of the formula (VIII) 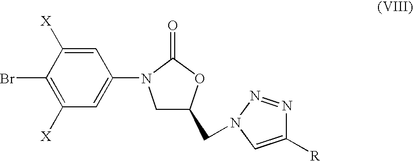
from a compound of the formula (VII), 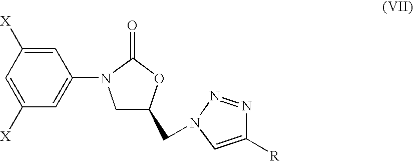
wherein each X is independently H or F and R is selected from hydrogen, halogen, cyano, methyl, cyanomethyl, fluoromethyl, difluoromethyl, trifluoromethyl and —Si[(1-4C)alkyl]₃;
said process comprising treatment of a solution of the compound of formula (VII) with bromine.

2. A process as claimed in claim 1, wherein the bromine is generated in situ from a bromate, a bromide and acid.

3. A process for preparing a compound of the formula (VIII) as defined in claim 1 from a compound of the formula (VII) as defined in claim 1, said process comprising treatment of a solution of the compound of formula (VII) with a bromate, a bromide and acid.

4. A process as claimed in claim 2, wherein the bromate is an alkali metal bromate.

5. A process as claimed in claim 2, wherein the bromide is provided by hydrobromic acid.

6. A process as claimed in claim 2, wherein hydrobromic acid provides the bromide and the acid.

7. A process for forming a compound of the formula (VIII) as defined in claim 1 from a compound of the formula (VII) as defined in claim 1, said process comprising treating a solution of the compound of formula (VII) with an alkali metal bromate, and hydrobromic acid.

8. A process according to claim 7 comprising:
a) treating a solution of the compound of formula (VII) in a mixture of water and a suitable organic solvent with aqueous hydrobromic acid; and
b) adding an aqueous solution of an alkali metal bromate.

9. A process according to claim 8 comprising the additional step of (c) adding a solution of sodium metabisulfite to react with any excess bromine.

10. A process according to claim 9 comprising the additional step of (d) isolating the product compound of the formula (VIII).

11. A process according to claim 10 wherein the step of isolating the product compound of formula (VIII) is performed by heating the mixture resulting from step c) until any solid has dissolved and then cooling the solution until the compound of the formula (VIII) crystallises.

12. A compound of the formula (VIII) 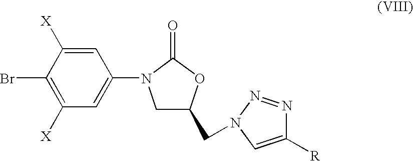
wherein each X is independently H or F; and
R is selected from hydrogen, halogen, cyano, methyl, cyanomethyl, fluoromethyl, difluoromethyl, trifluoromethyl and —Si[(1-4C)alkyl]₃.

13. A compound according to claim 12, wherein R is selected from hydrogen, halogen or methyl.

14. A compound according to claim 12 wherein R is hydrogen.

15. A compound according to claim 12 wherein at least one X is F.

16. A compound according to claim 15 wherein both X are F.

17. A compound according to claim 12 wherein one X is H and the other is F;
and R is hydrogen.
